(19) United States
(12) Patent Application Publication
(10) Pub. No.: US 2005/0203182 A1
(43) Pub. Date: Sep. 15, 2005

(54) Emulsification of lanolin

(75) Inventor: John Shortis, Maroochydore, Queensland (AU)

Correspondence Address: NIXON & VANDERHYE, PC, 901 NORTH GLEBE ROAD, 11TH FLOOR, ARLINGTON, VA, 22203, US

(73) Assignee: Supreme Protector Ireland Ltd., Country, Kildare (IE)

(21) Appl. No.: 10/515,577

(22) PCT Filed: May 22, 2003
(86) PCT No.: PCT/AU03/00619

§ 371 Date: Jan. 5, 2005

(30) Foreign Application Priority Data
May 22, 2002 (AU) PS 2476
Jun. 19, 2002 (AU) PS 3053
Nov. 13, 2002 (AU) 2002952616

(51) Int. Cl. C11D 3/386; A61K 31/225

(52) U.S. Cl. 514/547; 162/179; 510/353

(57) ABSTRACT

A process for emulsifying lanolin comprising blending a mixture of lanolin, a diamine emulsifier and water wherein the mixture has pH in the range of from 2 to 7 and wherein the mixture is at a temperature of at least 40° C. wherein the diamine emulsifier is present in the mixture in an amount in the range of from 1% by weight of lanolin to 5% by weight of lanolin, the water is present in an amount in the range of from 120% by weight of lanolin to 400% and wherein the mixture is blended under high shear conditions.

The present invention relates to the emulsification of lanolin. In particular, the present invention relates to a process for forming an oil-in-water emulsion of lanolin. Lanolin is well known for use as an emollient. Lanolin's non toxic properties render it suitable for use in a variety of applications. However, many potential uses of lanolin are impractical as lanolin is a highly viscous, waxy material and has a melting point of approximately 40° C. These physical properties of lanolin preclude its wide spread use.

Whilst there have been attempts to improve the physical properties of lanolin, such as by emulsification, to date there has not been found a practical and efficient means of producing a high lanolin content material that has suitable physical properties so as to permit the use of lanolin in a variety of applications that have hitherto been impractical to consider.

We have now found a process for the emulsification of lanolin to form a material with a high lanolin content that has physical properties suitable for a variety of applications. The process of the present invention may at least partially overcome the above-mentioned disadvantages or provide the consumer with a useful, or commercial, choice.

In one broad form, the present invention provides a process for emulsifying lanolin comprising blending a mixture of lanolin, a diamine emulsifier and water wherein the mixture has pH in the range of from 2 to 7 and wherein the mixture is at a temperature of at least 40° C. wherein the diamine emulsifier is present in the mixture in an amount in the range of from 1% by weight of lanolin to 5% by weight of lanolin, the water is present in an amount in the range of from 120% by weight of lanolin to 400% and wherein the mixture is blended under high shear conditions.

In a preferred form, the present invention provides a process for emulsifying lanolin comprising the steps of: heating lanolin to a temperature of at least 40° C. to 75° C.; adjusting the pH of the lanolin to a pH in the range of from 2 to 7; blending a diamine emulsifier into the lanolin wherein the diamine emulsifier is present in an amount in the range of from 1% by weight of lanolin to 5% by weight of lanolin; and gradually adding water in an amount in the range of from 120% by weight of lanolin to 400% by weight of lanolin under high shear conditions wherein the water is added at a temperature in a range of from 45° C. to 75° C.

The process of the present invention produces an oil-in-water emulsion having a high lanolin content. The oil-in-water emulsion is of sufficiently low viscosity at ambient temperatures to provide a convenient and efficient vehicle for the delivery of lanolin. The emulsion may be used as a concentrate that may be diluted with water or an aqueous solution without the need for further high speed mixing, simple stirring or mixing is sufficient. The emulsion permits lanolin to be used in a variety of applications that have hithertofor been considered to be impractical.

It will be appreciated that the term lanolin as used herein refers to a variety of materials that are derived from wool grease. The term lanolin herein includes wood wax ester, hydrous lanolin, anhydrous lanolin, wool fat, wool alcohol, adeps lanae, aloholes lanae, wool wax, wool grease, glossylan, golden dawn, nodorian, sparklelan. The nomenclature of lanolin and its derivatives often depends upon the context in which the term is used. Herein it is intended that the term lanolin include a broad range of materials that include wool grease and the products derived therefrom. Such products include lanolin alcohols and their derivatives as well as other lanolin derivatives. Wool grease may be recovered from wool processing operations such as scouring or solvent washing. Wool grease may be extracted from the liquor. The wool grease recovered from the liquor may be purified to remove impurities such as wool fibres, vegetable matter, pesticide residues, and other impurities. The wool grease may be refined and the refined wool grease is commonly known as lanolin. In the context of the present invention the term lanolin includes wool grease. Preferably the lanolin will have a reduced level of impurities relative to the wool grease and have any free fatty acids neutralised. The lanolin is preferably bleached and deodorised.

In the process of the present invention the lanolin is heated to a temperature of at least 40° C. Preferably to a temperature in the range of from 45° C. to 75° C. More preferably the lanolin is heated to a temperature in a range of from 50° C. to 70° C., most preferably to 60° C. At these temperatures the lanolin is liquid and may be readily handled by suitable processing equipment such as blenders, pumps and the like.

The process of the present invention may be integrated into existing wool scouring plants to process waste lanolin-containing materials. The hot lanolin-containing materials may be readily processed in accordance with the present invention. Water present with the lanolin-containing materials may be recycled, while hot, back to the start of the scouring process thereby removing or reducing some of the energy requirements necessary for the operation of the scouring plant. The integration of the process of the present invention into the operation of a wool scouring plant reduces the quantity of environmentally detrimental waste produced.

Lanolin may also be provided in 200 litre drums that are filled with lanolin extracted from a scouring process during the treatment of wool.

During the wool scouring process sulphuric acid is used. The sulphuric acid is neutralised after the scouring process by the addition of potash. An aqueous solution of potash may contaminate lanolin supplied from such sources. The aqueous solution containing potash will generally form a layer at the bottom of the drum of lanolin. In extracting the lanolin from the drum the lanolin needs to be heated. Preferably the heating is conducted in a manner that avoids boiling the aqueous solution containing potash and preventing excessive mixing of the potash throughout the lanolin. We have found that in extracting lanolin from drums using a slow or gentle heating whereby boiling of the aqueous layer containing potash lanolin may be extracted without undue contamination with potash. Furthermore drums of lanolin are often part filled during the scouring process, allowed to cool before further hot lanolin mixture is added to the drum. This may form multiple layers of aqueous solutions of potash. We have found that by the gentle heating of the drums of lanolin the layers of aqueous solutions of potash may be combined and the lanolin separated therefrom without undue mixing or contamination of the potash with the lanolin.

The pH of the lanolin is adjusted to a pH in the range of from 2 to 7. Preferably the pH is adjusted to be in the range of from 4 to 7. More preferably the pH is in the range of from 6 to 7, most preferably the pH of the lanolin is about 6. Typically, lanolin has a pH in excess of 7 and it is necessary to add an acid, preferably sulfuric acid, to adjust the pH of the lanolin to the desired level. It will be appreciated that other acids may be used to adjust the pH of the lanolin.

A diamine emulsifier is blended into the lanolin. Suitable diamine emulsifiers include diamines selected from the group consisting of octyl propylene diamine, lauryl propylene diamine, myristyl propylene diamine, palmityl propylene diamine, stearyl propylene diamine, behenyl propylene diamine, beef tallow propylene diamine, lauryl ethylene diamine, myristyl ethylene diamine and stearyl ethylene diamine. Preferably the diamine is a 1,3-diaminopropane. More preferably the diamine emulsifier is N-oleyl-1,3-diaminopropane.

The diamine emulsifier is blended into the lanolin in an amount of a range from 1% by weight of the lanolin to 5% by weight of the lanolin. Preferably the diamine emulsifier is present in the amount in a range of between 2% by weight and 3% by weight of the lanolin.

The lanolin is formed into an emulsion by gradually adding water under high shear conditions. Preferably the high shear conditions may be provided by a blender operating at in excess of 300 rpm. We have found that operating a mixer having a mixing head with a diameter of 50 mm a mixing speed in the range from 5000 to 6000 rpm is particularly preferred.

Whilst it is preferable to minimise the amount of water added in order to form the emulsion, we have found that it is necessary to add water in an amount in the range of from 120% to 400% by weight of the lanolin. Preferably the amount of water added is in the range of from 120% to 200%, more preferably 150% by weight of the lanolin. It will be appreciated that, dependant upon the particular application, it may be desirable to impart differing physical properties in the emulsion. This may be achieved by varying the quantity of water added.

The water is added at a temperature in the range of from 45° C. to 75° C. Preferably the temperature of the water is the same of that of the lanolin. It is preferred that the temperature of the water be in the range of from 50° C. to 70° C., more preferably at 60° C. Whilst the water may be at a slightly different temperature to the lanolin although it is preferred that the water be maintained at the temperature of the lanolin. We have found it preferable that the water be maintained at a temperature slightly higher than that of the lanolin rather than having the water at a temperature cooler than that of the lanolin.

The lanolin emulsion produced by the process of the present invention may be used in a variety of applications. For example, the lanolin emulsion may be used as a timber treatment so as to protect the timber.

The oil in water emulsion of lanolin may be applied to timber as a preservative. In order to cause the lanolin to impregnate the timber the boiling water emulsion of lanolin may be applied to the timber using hot water or steam techniques, vacuum or pressure application or by use of a solvent to assist in the penetration of the lanolin into the timber. Suitable solvents include alcohols.

We have found that the emulsion produced by the process of the present invention deters termites from attacking wood treated with the emulsion. The emulsion is able to penetrate deeply into the wood, and whether water or hydrocarbon is used as a carrier to take the lanolin emulsion into the wood, both will evaporate, leaving wax firmly entrenched in the wood fibres. Advantageously, the treatment of wood with the a mulch and of the present invention additionally offers protection against warping, splitting, dry rot, wet rot, and wood boring insects. Additionally, the anti-corrosive qualities in the emulsion will also protect nails, screws, and all metal fittings from rust and corrosion. In order for the lanolin emulsion to impregnate the timber, steam techniques, vacuum or pressure applications may be used. Once the timber is impregnated with the lanolin emulsion, it will cool and seal the timber from moisture entering.

The lanolin emulsion may be used to waterproof paper and cardboard products by impregnating the paper-based material by emersion in a hot lanolin emulsion.

We have found that the lanolin emulsion may be applied to porous materials such as timber and paper-based materials at the elevated temperatures, from the preferred blending temperatures of the method of the present invention up to the boiling point of the emulsion. We have found that using elevated temperatures assists in the impregnation of the porous materials with lanolin emulsion.

The lanolin emulsion may also be used as a rust proofing agent, soil/cement stabilising agent or as a paint additive.

The lanolin emulsion when used in cement compositions such as masonry blocks will result in increased water resistance of the cement product. The incorporation of the lanolin emulsion into the cement product does not seal the product and preclude water vapour from exiting the cement product but the water resistance of the lanolin assists in preventing water in liquid form from penetrating the cement product. Suitable cement products that can be formed from concrete having the lanolin emulsion incorporated therein include masonry blocks, syncrete and cement rendering. The incorporation of the lanolin emulsion into the cement product does not seal the product and preclude water vapour from exiting the cement product, but the water resistance of the lanolin assists in preventing water in liquid form from penetrating the cement product. This emulsion is highly effective in repelling moisture in applications such as building blocks, form concrete structures, patio tiles, roof tiles, mud brick construction etc. It may repel rising dampness in all concrete/brick or other structures.

The lanolin emulsion may also be used as a cutting compound for metals such as aluminium.

When used as an additive for paint the lanolin emulsion may provide lanolin in amounts in excess of those typically used in paints. For example in a water based paint 20% to 25% of lanolin emulsion may be added and the paint, once dried has been found to have improved washability characteristics as well as improved elasticity. The lanolin emulsion may be added to calcemine paints as well as to polyurethane based paints.

Another application for the lanolin emulsion is in the preparation of road base. The lanolin emulsion may be used to provide water resistance to the road base. One method of preparing a road base is to introduce microbes into the disturbed earth and allow the microbes to consume the nutrients in the earth. After about 90 days the nutrients are exhausted and the microbes die off. Whilst we have found that the lanolin emulsion may detrimentally effect of the microbes, the incorporation of the lanolin emulsion into the nutrient poor earth provides a particularly stable, water resistant road base. The incorporation of the level of emulsion into soil, sand or other aggregate will greatly reduce maintenance costs on all types of roads or road construction, and greatly reduce erosion problems. The lanolin emulsion can be mixed with road base materials prior to compaction, Which will dramatically repel moisture or water, and fully stabilize and bind the road materials. The lanolin emulsion may greatly reduce costs for road maintenance.

Another application for the process of the present invention is in soil remediation. In many countries environmental laws do not prevent wool scouring plants from discarding lanolin-containing materials. The present invention may be used to treat contaminated soils by emulsifying the lanolin.

The present invention will now be illustrated with reference to the following non-limiting examples.

### EXAMPLE 1

40 ml of wool grease was tested to determine the pH level. The pH of the wool grease was adjusted to 6.5 by the addition of sulphuric acid. The wool grease was heated to a temperature of 60° C. 1.5 ml of Redicote E-16 (ex Akzo Nobel) was added to the wool grease with stirring. The temperature of the wool grease was maintained at 60° C.

60 ml of water was heated to 60° C. and slowly added to the wool grease in a high speed blender over a period of approximately two minutes. The mixer had a 50 ml diameter mixing head rotating at a speed in the range of between 5000 to 6000rpm.

### EXAMPLE 2

Similar experiments to that conducted in example 1 were conducted using varying ratios of wool grease to water and at different levels of pH. The diamine emulsifier used was N.O.S (oleylpropylene-diamine) in the amount of 1.5 ml per 40 ml of wool grease. The results are shown in table 1 below.

| Wool Grade | Water Content | PH | Temperature | Results |
|---|---|---|---|---|
| 40% | 60% | 6-7 | 60° C./50° C. | Emulsion formed |
| 30% | 70% | 6-7 | 60° C./50° C. | Emulsion formed |
| 90% | 80% | 6-7 | 60° C./50° C. | Emulsion formed |
| 40% | 60% | 4-5 | 40° C./50° C. | Emulsion formed |
| 30% | 70% | 4-5 | 40° C./50° C. | Emulsion formed |
| 90% | 80% | 4-5 | 40° C./50° C. | Emulsion formed |

### EXAMPLE 3

The housing of a 240 V AC electric drill was removed. The drill was immersed in a Lanolin-hydrocarbon based mixture, removed, and left to dry for a period of three days. The drill was re-assembled, and placed in a plastic container of water totally submerged. Power was turned on, and the drill continued to operate without failure.

### EXAMPLE 4

A sheet of cardboard was prepared, and an emulsion water based paint was painted onto the right hand section of the cardboard. (SECTION A) An opened ended jar was glued to the painted section. The lanolin emulsion was added to the paint—20% by weight and mixed. The mixture was painted on to the left side of the cardboard (SECTION B)

An open ended jar was glued to the painted section. Both jars were then filled with water, and examined 24 hours later. Section A cardboard was examined and showed dampness underneath where the jar was positioned after a period of twenty four hours. Section B cardboard was examined, with no visible sign of dampness. The cardboard was examined for a period of 14 days, with no sign of any dampness in that period. Over a period of six months, no leakage occured.

### EXAMPLE 5

### Methodology

| | |
|---|---|
| Timber Species | Radiator Pine |
| Treatment | lanolin emulsion according to Example 1 |

(6) Blocks cut from same piece of timber—90-45 mm

(2) Blocks were immersed in the formulae till saturation point and then weighed. The (2) blocks were then allowed to dry and weighed to determine retension of active component.

A 25% Active was recorded.

The (4) remaining blocks were left untreated.

| | |
|---|---|
| Treated blocks (2) | refer to as (A) |
| Untreated blocks (2) | refer to as (B) |
| Untreated blocks (2) | refer to as (C) |

### Test Procedure

Blocks (A) and (B) were placed in an active termite area of fenceline and covered with existing mulch to a depth of 60 mm

### Results

After 6 months blocks (A) and (B) were uncovered for examination. Blocks (A) were untouched by termites, and in the same condition as before test. Blocks (B) were completely devoured by termites to egg shell thickness to the walls and showing rot.

Blocks (A) and (B) were returned with the addition of Blocks (C), covered again to a depth of 60 mm with existing mulch.

### Results

After another 7 months Blocks (A) (B) (C) were uncovered and examined. Blocks (A) were discoloured by moisture, but showed no signs of termite attack or rot. Blocks (B)—What remained of the blocks had severe rot and decomposition. Blocks (C) Termites had hollowed out the blocks, with rot well advanced.

1. A process for emulsifying lanolin comprising blending a mixture of lanolin, a diamine emulsifier and water wherein the mixture has pH in the range of from 2 to 7 and wherein the mixture is at a temperature of at least 40° C. wherein the diamine emulsifier is present in the mixture in an amount in the range of from 1% by weight of lanolin to 5% by weight of lanolin, the water is present in an amount in the range of from 120% by weight of lanolin to 400% and wherein the mixture is blended under high shear conditions.

2. A process according to claim 1 wherein the diamine emulsifier is selected from the group consisting of octyl propylene diamine, lauryl propylene diamine, myristyl propylene diamine, palmityl propylene diamine, stearyl propylene diamine, behenyl propylene diamine, beef tallow propylene diamine, lauryl ethylene diamine, myristyl ethylene diamine and stearyl ethylene diamine.

3. A process according to claim 1 wherein the diamine emulsifier is N-oleyl-1,3-diaminopropane.

4. A process according to claim 1 wherein the diamine emulsifier is present in the amount in a range of between 2% by weight and 3% by weight of the lanolin.

5. A process according to claim 1 wherein the lanolin is heated to a temperature in the range of from 45° C. to 75° C.

6. A process according to claim 1 wherein the lanolin is heated to a temperature of 60° C.

7. A process according to claim 1 wherein the pH is adjusted to be in the range of from 6 to 7.

8. A process according to claim 1 wherein the amount of water present is in the range of from 120% to 200%.

9. A process according to claim 1 wherein the amount of water present is 150% by weight of lanolin.

10. A process for emulsifying lanolin comprising the steps of:
heating lanolin to a temperature of at least 40° C. to 75° C.;
adjusting the pH of the lanolin to a pH in the range of from 2 to 7;
blending a diamine emulsifier into the lanolin wherein the diamine emulsifier is present in an amount in the range of from 1% by weight of lanolin to 5% by weight of lanolin; and
gradually adding water in an amount in the range of from 120% by weight of lanolin to 400% by weight of lanolin under high shear conditions wherein the water is added at a temperature in a range of from 45° C. to 75° C.

11. A process according to claim 10 wherein the diamine emulsifier is selected from the group consisting of octyl propylene diamine, lauryl propylene diamine, myristyl propylene diamine, palmityl propylene diamine, stearyl propylene diamine, behenyl propylene diamine, beef tallow propylene diamine, lauryl ethylene diamine, myristyl ethylene diamine and stearyl ethylene diamine.

12. A process according to claim 10 wherein the diamine emulsifier is N-oleyl-1,3-diaminopropane.

13. A process according to claim 10 wherein the diamine emulsifier is present in the amount in a range of between 2% by weight and 3% by weight of the lanolin.

14. A process according to claim 10 wherein the lanolin is heated to a temperature in the range of from 45° C. to 75° C.

15. A process according to claim 10 wherein the lanolin is heated to a temperature of 60° C.

16. A process according to claim 10 wherein the pH is adjusted to be in the range of from 6 to 7.

17. A process according to claim 10 wherein the amount of water present is in the range of from 120% to 200%.

18. A process according to claim 10 wherein the amount of water present is 150% by weight of lanolin.

19. A wool scouring process including the process according to claim 1.

20. A lanolin emulsion produced by the process of claim 1.

21. A process for the treatment of wood comprising impregnating the wood with a lanolin emulsion according to claim 20.

22. A process for waterproofing a paper or cardboard product comprising applying a lanolin emulsion according to claim 20 to the paper or cardboard product.

23. A cement composition comprising a lanolin emulsion according to claim 20.

24. A road base comprising a lanolin emulsion according to claim 20.
